(19) United States
(12) Patent Application Publication
(10) Pub. No.: US 2018/0078181 A1
(43) Pub. Date: Mar. 22, 2018

(54) HEALTH WEARABLE USING SMART ENERGY HARVESTING

(71) Applicant: KONINKLIJKE PHILIPS N.V., EINDHOVEN (NL)

(72) Inventors: JOHN CRONIN, BONITA SPRINGS, FL (US); CHRISTOPHER HUFFINES, WILLISTON, VT (US)

(21) Appl. No.: 15/559,439

(22) PCT Filed: Mar. 18, 2016
(86) PCT No.: PCT/IB2016/051523

§ 371 Date: Sep. 19, 2017

Related U.S. Application Data
(60) Provisional application No. 62/135,873, filed on Mar. 20, 2015.

(51) Int. Cl. A61B 5/11; G06F 19/00; A63B 24/00; A61B 5/00

(52) U.S. Cl. CPC A61B 5/1118; G06F 19/3406; A63B 24/0062; A61B 5/0205; A63B 24/0006; G06F 19/3481; A61B 5/681

(57) ABSTRACT

A wearable device with an energy harvesting circuit calculates a trendline based on a plot of multiple calorie amounts, each calorie amount associated with an energy amount. Each calorie amount is based on one or more sensor measurements over a particular time period corresponding to a fitness activity from one or more sensors of the wearable device. Each energy amount is an amount of energy produced by the energy harvesting circuit during the time period corresponding to the fitness activity. The wearable device uses the trendline to determine how many calories the user should burn in order for the energy harvesting circuit to produce enough electric charge to charge the wearable device to a predetermined battery charge level and outputs a user alert based on this amount of calories.

### BACKGROUND

### Field of Disclosure

The present description generally relates to a wearable technology and, more specifically, to wearable devices that use energy harvesting circuits to replenish stored electric charge.

### Description of the Related Art

Wearable technology may include any type of mobile electronic device that can be worn on the body, attached to or embedded in clothes and accessories of an individual and currently exist in the consumer marketplace. Processors and sensors associated with the wearable technology can display, process or gather information. Such wearable technology has been used in a variety of areas, including monitoring health data of the user as well as other types of data and statistics. These types of devices may be readily available to the public and may be easily purchased by consumers. Examples of some wearable technology in the health arena include the FitBit, the Nike Fuel Band, the Jawbone Up, and the Apple Watch.

Typically, a wearable device can be used to gather data about the user. For example, a wearable device can use one or more sensors to monitor health parameters of a user. Such sensor measurements can sometimes be used to calculate metrics such as calories burned by the user.

Each sensor or other component of a wearable device generally draws power from a battery. Sometimes, a wearable device may have some understanding of how much battery power a particular component generally uses. Wearable devices are typically small devices, which in turn means that their batteries are small, which in turn means that they must be charged often. Sometimes, wearable devices must have their batteries recharged after less than 24 hours of use. Thus, battery life is often a significant barrier to wearable use.

Energy-harvesting circuits exist that can generate electric energy to power a battery by generating energy from the movements of the user of the wearable device such as by harvesting energy from the user swinging their arms if the wearable device takes the form of a smart watch or bracelet, or from solar power, or from heat such as body heat. Such energy-harvesting circuits can be integrated into a wearable device in order to provide additional electrical power to the battery of the wearable device throughout the day.

However, even if a wearable device includes an energy-harvesting circuit, a user of such a wearable device might not know what sort of specific actions they can take, or how much of a specific action they can take, in order to help recharge the wearable device's battery by a specific amount.

Therefore, there is a need for an improved wearable device with an energy harvesting circuit.

### SUMMARY

The present disclosure relates to a wearable device which is configured to receive energy harvesting information and utilize such information as a basis for additional related information for display and presentation to the user. The wearable device in various configurations is operable to receive information from the one or more energy harvesting circuits which are able to detect, over a period of time, calorie amounts or other related fitness activity. The energy or calorie amount may be correlated with the amount of energy produced by the energy harvesting circuit. The wearable device may be operable to develop a trendline to determine how many calories a user should burn in order to produce enough electric charge to charge the wearable device to a predetermined battery level based upon historical trendline information and determination for the user or a plurality of users.

In various aspects, the wearable device of the present disclosure may be operable to utilize the sensors to generate information related to calories used during physical activity. The device may also be operable to also determine the energy developed by an energy harvesting circuit during the physical activity and subsequently store such information related to calories expended and related energy produced by the energy harvesting circuit into a database. In further aspects, the wearable device described in the present disclosure may be operable to determine a trendline of best fit predicting the number of calories required to harvest a particular amount of energy. Such information may then be utilized by the wearable device to determine the necessary amount of activity required to recharge the battery of the wearable device to a predetermine level. The user may then be informed of such determination through a display on the wearable device or associated display so that the user is aware of the number of calories the user will need to burn to generate sufficient charge so that the device is charged to a desirable level.

The present disclosure further includes in various configurations a wearable device which incorporates at least one energy harvesting circuit in combination with other sensors on the wearable device which can detect additional information related to the user.

Embodiments of the present disclosure includes systems and methods directed towards smart energy harvesting in a wearable device and display of relevant determined information for the user in order to allow the user to more readily determine fitness activity and energy produced, for example.

In one aspect, a method for using smart energy harvesting begins with the wearable device operable to calculate a trendline based on historical data stored in a memory of a wearable device. In some aspects, the historical data may include a plurality of calorie amounts, where each calories amount of the plurality of calorie amounts is calculated based on one or more sensor measurements generated by one or more sensors of the wearable device during a sensed time period. In other aspects, each calorie amount of the plurality of calorie amounts may also be associated with an energy amount, the energy amount indicating an amount of energy generated by an energy harvesting circuit during the calorie amount's sensed time period. In various embodiments and implementations, the wearable device is configured and is operable to determine a current battery charge level of a battery of the wearable device. The wearable device may determine a charge difference indicating a required amount of electric charge from the energy harvesting circuit to increase the current battery charge level to a predetermined battery charge level of the battery of the wearable device. The predetermined battery charge level may inclusively be between the current battery charge level and a full battery charge level of the battery of the wearable device. The wearable device may be configured to calculate a calorie requirement indicating a number of calories that, according to the trendline, should generate the charge difference through the energy harvesting circuit. The wearable device may then, in some implementations, generate an alert based on the calorie requirement.

In some implementations, the present disclosure relates to a system and a method for implementing on a wearable device; calculating a trendline based on historical data stored in a memory of a wearable device; wherein the historical data includes a plurality of calorie amounts. In some aspects, each calories amount of the plurality of calorie amounts is calculated based on one or more sensor measurements generated by one or more sensors of the wearable device during a sensed time period and each calorie amount of the plurality of calorie amounts is also associated with an energy amount, the energy amount indicating an amount of energy generated by an energy harvesting circuit during the calorie amount's sensed time period. Further aspects include determining a current battery charge level of a battery of the wearable device and determining a charge difference indicating a required amount of electric charge from the energy harvesting circuit to increase the current battery charge level to a predetermined battery charge level of the battery of the wearable device, wherein the predetermined battery charge level is inclusively between the current battery charge level and a full battery charge level of the battery of the wearable device. The wearable device may further be operable for calculating a calorie requirement indicating a number of calories that, according to the trendline, should generate the charge difference through the energy harvesting circuit; and generating an alert based on the calorie requirement.

In some implementations a system is provided that includes a wearable device having at least one sensor, the wearable device further having at least one energy harvesting circuit electrically connected to a power storage unit or similar battery type device. The system may include at least one processor connected to a memory and having instructions configured to determine calories used of user and energy developed during a determined time period based upon information received from the at least sensor and the at least one energy harvesting circuit; record the determined calories used and energy developed in a historical charging database; generate a best fit line from the historical charging database; determine a power charge level for the power storage unit of the wearable device; determine an amount of energy needed to charge the power storage unit to a predetermined level based on the best fit line, power charge level; provide the determined amount of energy needed to a display.

In various implementations, the system may be further configured to present to the display a selectable list of the at least one sensor; receive instructions to disable at least one of the sensors and determine energy savings from the at least one disabled sensor. Alternatively, the system may also be configured to present the determined energy savings to the display. Additionally, the sensor of the system may include a plurality of sensors which monitor health parameters of a wearer of the wearable device. In other implementations, the at least one energy harvesting circuit generates energy during physical activity of a wearer of the wearable device.

In other implementations, a method for providing energy harvesting information for a wearable device is described which includes determining calories used of user and energy developed during a determined time period based upon information received from at least sensor on the wearable device and at least one energy harvesting circuit on the wearable device; determining a power charge level for a power storage unit of the wearable device; calculating an amount of energy needed to charge the power storage unit to a predetermined level and displaying the determined amount of energy needed.

In some implementations, the method may further include saving the calories used and the energy developed during the predetermined time period. Still other aspects of the method may further include calculating the amount of energy needed to charge the power storage unit based upon the saved calories used and energy developed.

In some embodiments, the method may provide for calculating the amount of energy based upon a wearer of the wearable device past activity and even further embodiments may include calculating the amount of energy based upon a trendline created from the saved calories used and energy developed.

Various aspects of the disclosure may further implement calculating the amount of energy based upon a wearer's current activity level or alternatively based upon a wearer's historical activity level.

In some implementations, the method may further include presenting to a display of the wearable device a selectable list of the at least one sensor, receiving instructions to disable at least one of the sensors, determining energy savings from the at least one disabled sensor, and calculating a second amount of energy needed to charge the power storage unit to the predetermined level. Additionally, some embodiments of the method may further comprising presenting to the display the second amount of energy. The method may, in additional implementations, further include presenting to the display a time period related to generating the second amount of energy.

Techniques, methods and apparatus described herein may give rise to a variety of technical advantages. For example, the wearable device described in the present disclosure may create an historical charging database which correlates both calories used, time and energy developed by an energy harvester. Using such information, the wearable device, system and method may create a best fit line using the data for calories burned to energy from an energy harvester circuit and store the best fit line in the historical charging database. With such information the user may directly obtain relevant information regarding the wearable device, how much energy is required (e.g. how much activity is needed) to charge the battery to full. Once determined, the determined relevant information may be presented to the user from a display on the wearable device. Furthermore, advantages include allowing the user to selectively determine energy savings form a component power database and selectively be advised of the effect turning off such components will have on the required energy for full battery charge.

Other implementations may include a non-transitory computer readable storage medium storing instructions executable by a processor (e.g., a central processing unit (CPU) or graphics processing unit (GPU)) to perform a method such as one or more of the methods described above. Yet another implementation may include a system of one or more processors operable to execute stored instructions to perform a method such as one or more of the methods described above.

It should be appreciated that all combinations of the foregoing concepts and additional concepts described in greater detail herein are contemplated as being part of the subject matter disclosed herein. For example, all combinations of claimed subject matter appearing at the end of this disclosure are contemplated as being part of the subject matter disclosed herein.

### BRIEF DESCRIPTION OF THE DRAWINGS

FIG. 1 illustrates an exemplary wearable device with exemplary energy harvesting elements.

FIG. 2A is an exemplary scatter plot with an exemplary trendline, the scatter plot charting exemplary “calories burned” measurements against exemplary “energy from energy harvester for charging” measurements.

FIG. 2B is an exemplary chart illustrating an exemplary full battery charge level, an exemplary current battery charge level, and a distance between these representing electrical charge required from an energy harvester circuit for an exemplary battery at the illustrated current battery charge level to reach the full battery charge level.

FIG. 2C is an exemplary chart illustrating an exemplary number of burned calories that is extrapolated from the trendline of FIG. 2A to produce the electrical charge required from an energy harvester circuit of FIG. 2B for the battery to reach the full battery level.

FIG. 3 is a flow diagram illustrating an exemplary operation of a wearable software as executed by an exemplary wearable device.

FIG. 4 illustrates an exemplary energy harvesting graphical user interface (GUI) as executed by an exemplary wearable device.

FIG. 5 illustrates an exemplary wearable output graphical user interface (GUI) as executed by an exemplary wearable device.

FIG. 6 illustrates an exemplary computing device architecture that may be utilized to implement the various features and processes described herein.

FIG. 7 illustrates an exemplary historical charging database that may be stored in the memory of an exemplary wearable device.

FIG. 8 illustrates an exemplary component power database that may be stored in the memory of an exemplary wearable device.

FIG. 9 illustrates an exemplary overall method of the present embodiments as described herein.

### DETAILED DESCRIPTION

Several implementations of the technology described herein are provided with reference to the appended drawings. The following description and drawings are illustrative of the various embodiments and implementations and are not to be construed as limiting. However, in certain instances, well-known or conventional details are not described in order to provide a concise discussion of the aspects and embodiments described herein.

Reference in the specification to “one embodiment” or “an embodiment” or an implementation means that a particular feature, structure, or characteristic described in conjunction with the embodiment can be included in at least one embodiment of the disclosure. The appearances of the phrase “in one embodiment” and the like in various places does not necessarily refer to the same embodiment.

Embodiments described herein relate to a wearable device with an energy harvesting circuit, the device having a processor or similar structure programmed with instructions that may calculate a trendline based on a plot of multiple calorie amounts, each calorie amount associated with an energy amount. Each calorie amount may be based on one or more sensor measurements over a particular time period (e.g. corresponding to a fitness activity) from one or more health/fitness sensors (e.g., accelerometers, heart rate sensors) of the wearable device. Each energy amount is an amount of energy produced by the energy harvesting circuit during the time period (e.g., corresponding to the fitness activity). The wearable device may be configured to utilized such trendline to determine how many calories the user should burn in order for the energy harvesting circuit to produce sufficient electric charge to charge the wearable device to a predetermined battery charge level (e.g., maintaining or increasing current battery level), and outputs a user alert based on this amount of calories.

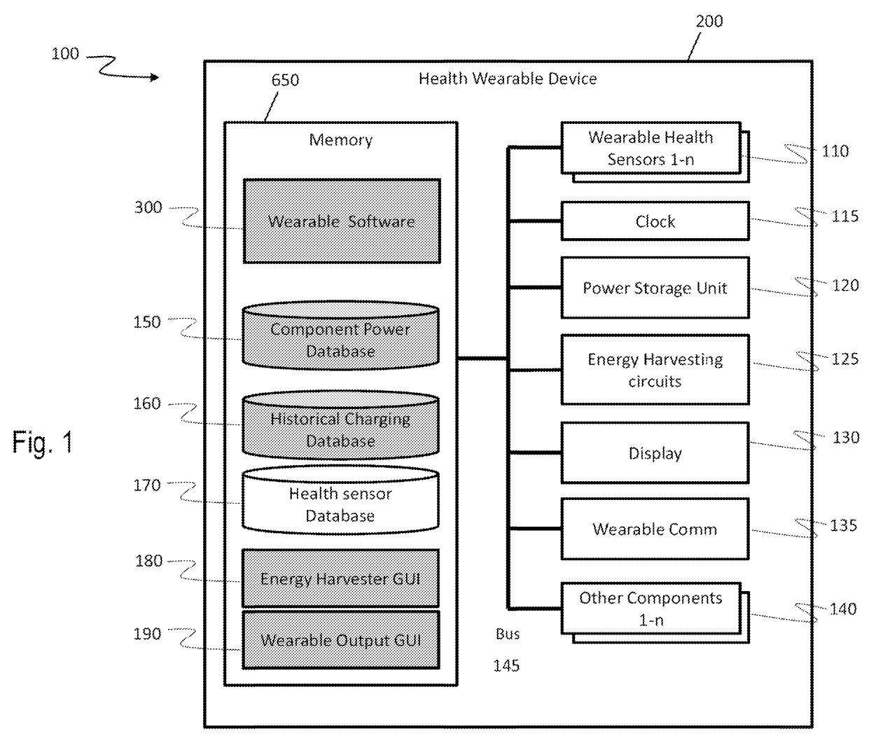

FIG. 1 illustrates an exemplary wearable device 200 for the system 100 with exemplary energy harvesting elements variously described herein in the multiple implementations and embodiments.

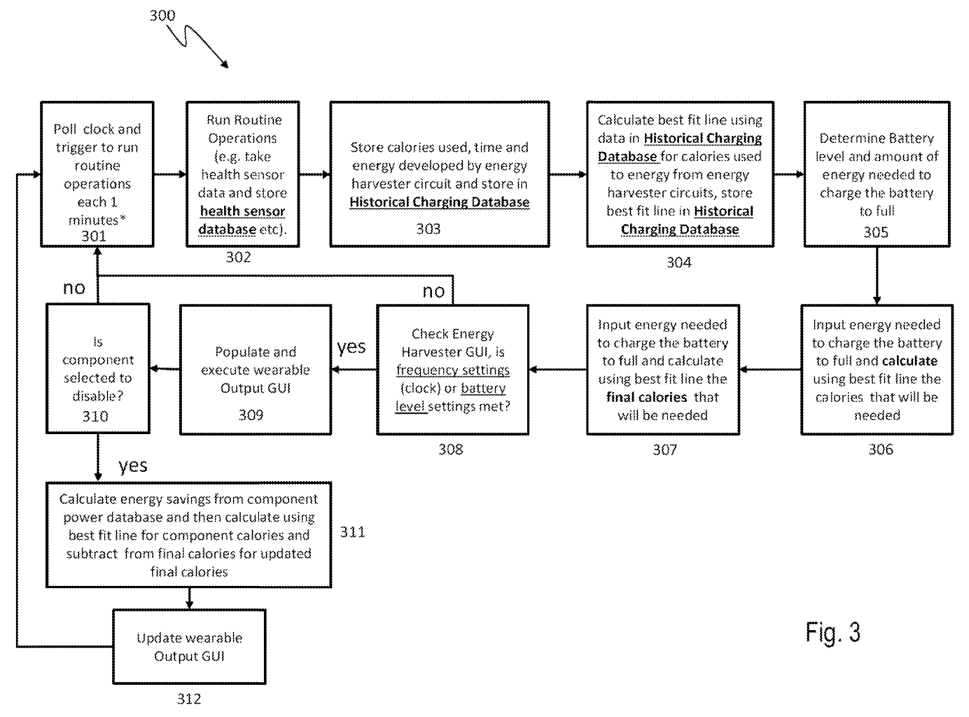

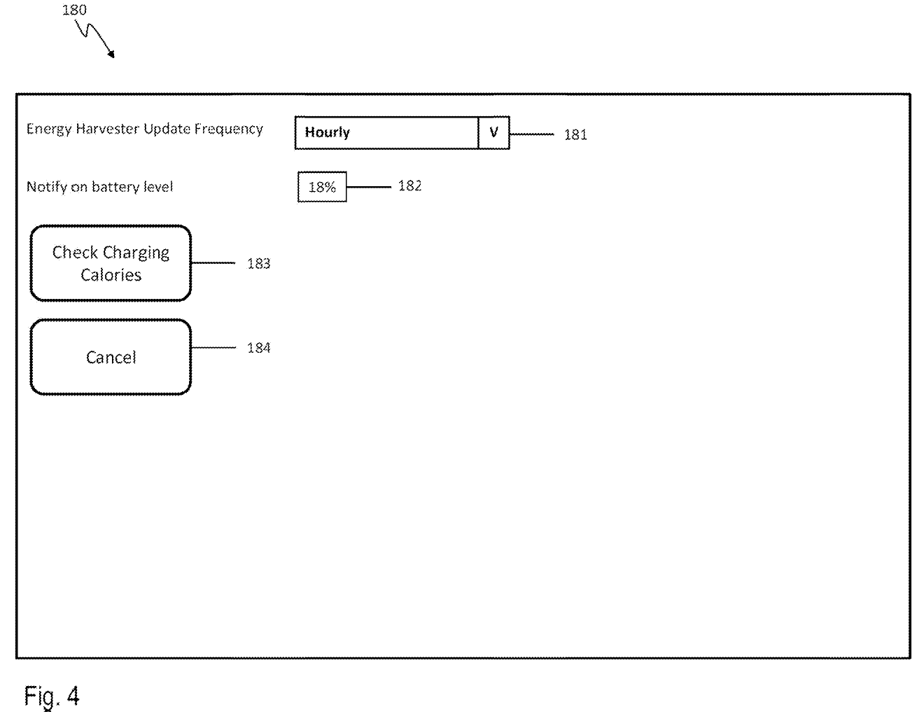

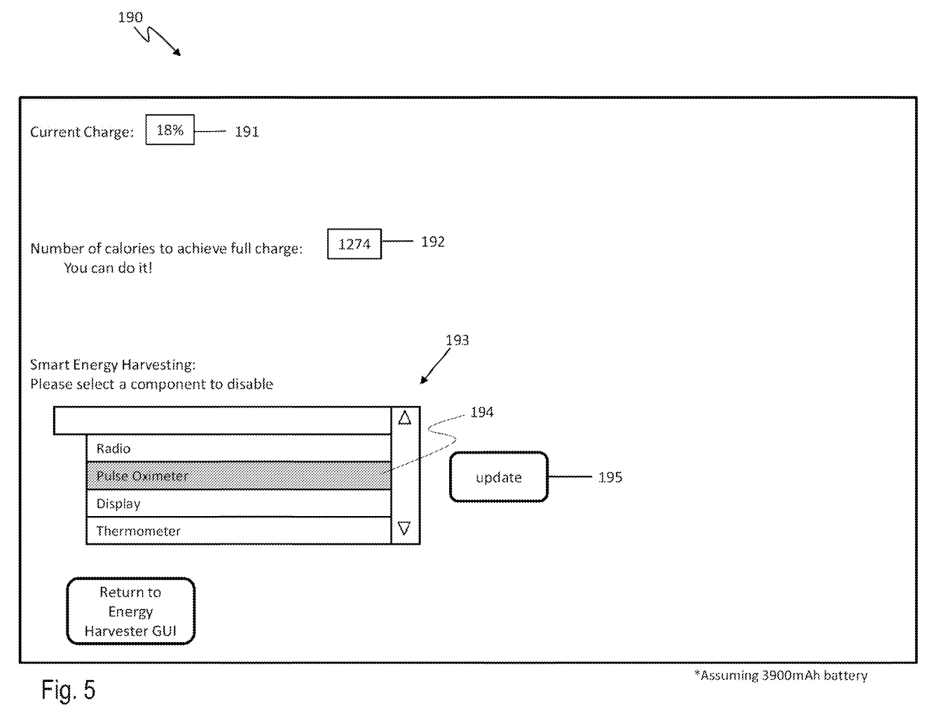

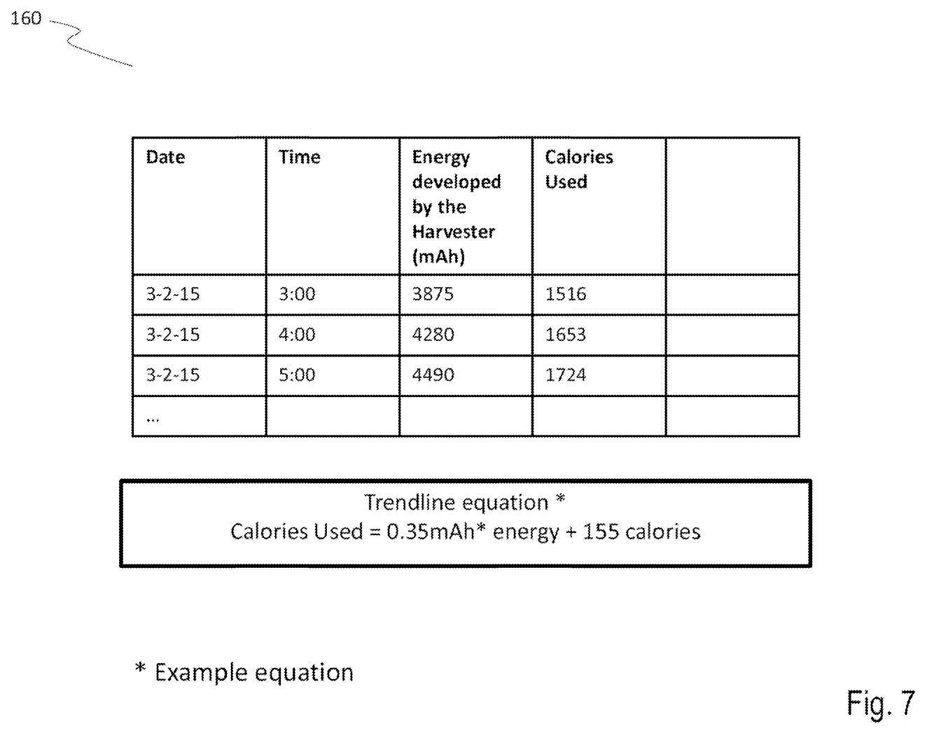

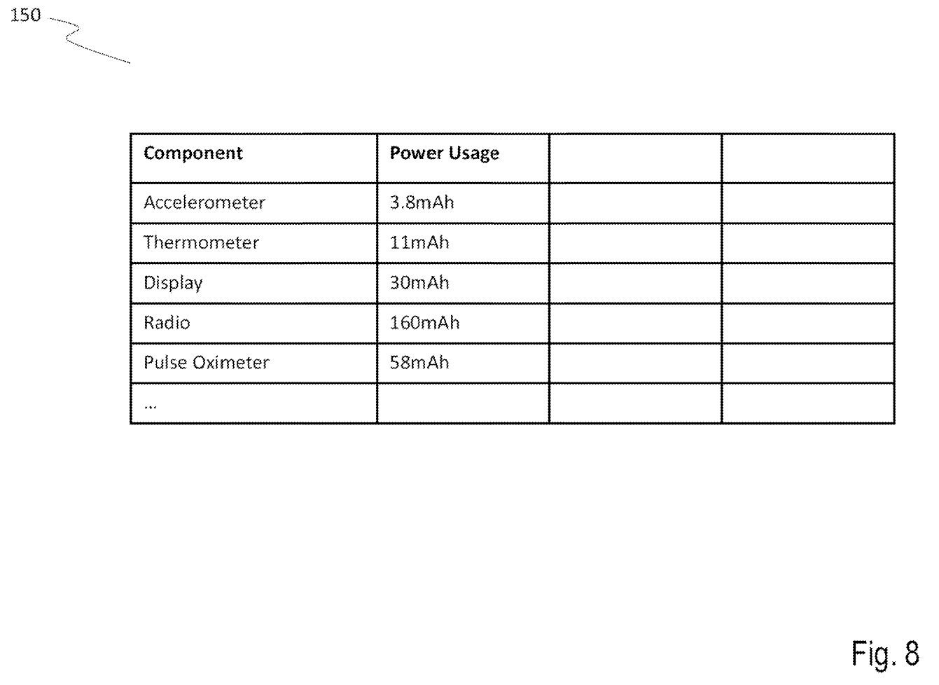

The wearable device 200 may include a number of components which perform aspects of the described features. For example, the wearable device may include one or more wearable device health/body/fitness sensors 1-n 110, a clock 115, a power storage unit 120, one or more energy harvesting circuits 125, a display 130, a memory 650, a communication module (“wearable comm”) 135, and a variety of other components 1-n 140. These components may be communicatively coupled at a single bus 145, or may alternatively be connected in a more disjointed manner. The memory of the wearable device may include a wearable software 300 (see e.g., FIG. 3), a component power database 150 (see e.g., FIG. 8), a historical charging database 160 (see e.g., FIG. 7), a health sensor database 170, an energy harvester graphical user interface 180 (“GUI”) (see e.g., FIG. 4), a wearable output GUI 190 (see e.g., FIG. 5), and various other software elements. The wearable device architecture illustrated in FIG. 1 should be interpreted as illustrative rather than limiting, and other embodiments may include additional or different components and/or elements stored in memory, and/or may lack illustrated components or elements stored in memory.

For example, the system 100 of the health wearable device may include multiple processors which are in communication with the memory and various components and may integrate various displays either on the wearable or projected therefrom or viewable separately. Further, the wearable device may further include various communication functionality which allows the multiple memory storage to be segmented separate and apart from the memory 650. For example, in some embodiments, the component database, historical charging database or health sensor database may be available remotely in a remote storage and the wearable device may communication with the remote storage via wireless or other communication protocols and techniques.

The communication port/module 135 of the wearable device 200 may be a wired connection module such as a USB port module, a FireWire port module, a Lightning port module, a Thunderbolt port module. The communication module may also be a physical connection module such as one that communicates through a direct physical contact of one or more conductive leads of the wearable device to one or more conductive leads of another device or power source. Alternatively or in combination, the communication module may further be a wireless connection module such as a Wi-Fi connection module, a 3G/4G/LTE cellular connection module, a Bluetooth connection module, a Bluetooth low energy connection module, Bluetooth Smart connection module, a near field communication module, a radio wave communications module, a magnetic induction power transmitter/receiver, or a magnetic resonance power transmitter/receiver.

The one or more wearable device sensors 110 of the wearable device 200 may include sensors for measuring blood pressure, heart rate, pulse (e.g., a pulse oximeter), body temperature (e.g., a thermometer), blood sugar, blood glucose (e.g., a glucometer), acceleration (e.g., an accelerometer), insulin, vitamin levels, respiratory rate, heart sound (e.g., a microphone), breathing sound (e.g., a microphone), movement speed (e.g., an accelerometer), steps walked or ran (e.g., a pedometer), skin moisture, sweat detection, sweat composition, nerve firings (e.g., an electromagnetic sensor), or similar health measurements. In some embodiments, additional sensors may also measure allergens, air quality, air humidity, air temperature, and similar environmental measurements.

The power storage unit 120 may be any type of unit capable of storing power over a period of time, such as a rechargeable battery (e.g., Nickel Cadmium or “NiCd”, Nickel Metal Hydride or “NiMH”, Lithium Ion or “Li Ion”, Sealed Lead Acid or “SLA”), a capacitor, a potential-energy-based power storage unit, a chemical-energy-based power storage unit, a kinetic-energy-based power storage unit, or some combination thereof. Reference to a “battery” of the wearable device herein should be understood to refer to any of these types of power storage units. The power storage unit 120 may also include sensors and processors. For example, some lithium ion rechargeable batteries include sensors that limit discharge and recharging of the battery in manners that preserve/increase the battery's lifespan.

The wearable device may include one or more energy harvesting circuits 125 which each motion-based (e.g., piezoelectric circuits), heat-based (e.g., thermal generation), light-based (e.g., solar cells), or chemically-based (e.g., hydrogen cells).

The display 130 may be a touch-sensitive display (e.g., a capacitive multi-touch display) to allow a user to interact with a graphical user interface displayed through the display. The display can also be non-touch-sensitive, and any user interfaces described herein may instead be operated through physical/mechanical interface components such as buttons, radio buttons, levers, switches, wheels, sliders, touchpads, keyboards, mice, and other physical/mechanical interface elements embedded within or connected to the wearable device.

The other components 1-N 140 may include any other component that could reasonable fit into a wearable device, or be connected (in a wired or wireless fashion) to a wearable device. For example, the other components 1-N may include one or more speakers, one or more vibrators, one or more lights (e.g., light emitting diodes), one or more camera devices and/or one or more thermal sensors.

The memory 650 of the wearable device 200 may be any type of memory or storage component, including a flash memory (NOR flash or NAND flash), an electrically erasable programmable read-only memory (EEPROM), read-only memory (ROM), random access memory (RAM), dynamic random access memory (DRAM), a hard drive (HDD), an optical-disc-based memory, a memristor-based memory, or a tape-based memory.

The databases stored in the memory of the wearable device may be a different type of file than a traditional database. Reference to the term database or databases herein should be understood to include any data structure that can hold data about one or more entities, such as a database, a table, a list, a matrix, an array, an arraylist, a tree, a hash, a flat file, an image, a queue, a heap, a memory, a stack, a set of registers, or a similar data structure.

The wearable device could be primarily intended to be worn around a user's wrist (e.g., a watch or bracelet), neck (e.g., a necklace or scarf), arm (e.g, an armband or elbow brace), hand (e.g, a glove), finger (e.g., a ring), head (e.g., a hat or helmet or headband or headlamp), leg (e.g., a knee brace or leg holster or pair of pants), torso (e.g., a shirt or sweater or jacket), chest (e.g., a heart monitor chest band/patch, a respiratory monitor chest band/patch), pelvic area (e.g., an undergarment or a swimsuit or a jock strap), waist (e.g., a belt), foot (e.g., a shoe or sock or ankle brace), or another area of the user's body.

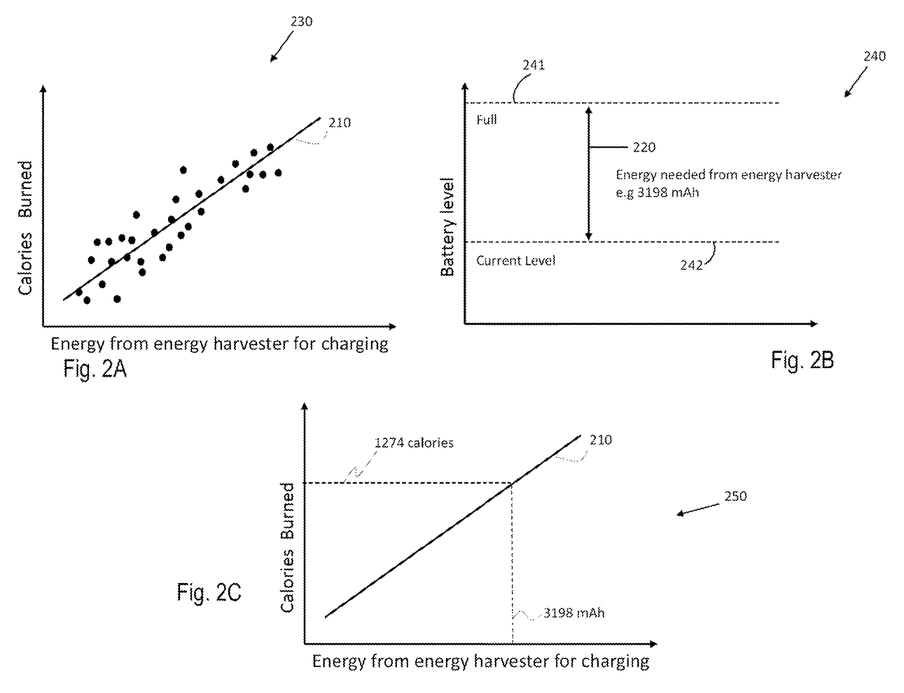

FIG. 2A is an exemplary scatter plot 230 with an exemplary trendline 210, the scatter plot charting exemplary “calories burned” measurements against exemplary “energy from energy harvester for charging” measurements. This calculation is performed by the wearable software of the wearable device (see e.g., FIG. 3).

In particular, each calorie amount may based on one or more sensor measurements over a particular time period (e.g. corresponding to a fitness activity) from the one or more health/body/fitness sensors 1-N (e.g. accelerometers, heart rate sensors) of the wearable device. Each energy amount is an amount of energy produced by the energy harvesting circuit during the time period (e.g., corresponding to the fitness activity). Each calorie amount and energy amount correspond to a single point in the scatter plot 230 of FIG. 2A.

The trendline 210 may be one of a variety of types of trendlines. The trendline may be a linear trendline, often referred to as a “best-fit line,” as pictured in FIG. 2A. The trendline may alternately be a curved trendline, such as a logarithmic trendline, a polynomial trendline, a power trendline, an exponential trendline, a moving average trendline, a sinusoidal trendline, a geometric trendline, or another type of curved trendline. The trendline may be calculated all at once or in a segmented fashion through calculation of localized trendline segments.

In various examples using the above implementations, the trend line may resolve to a formula for determination of calories used as a basis of production units of energy (such as for example 0.35 mAh) times the energy+a predetermined baseline calorie amount. For example, in the example of the plot 230, determination of calories used may be equated to 0.35 mAh×energy+155 calories.

The trendline 210 may be calculated using a variety of algorithms, including a total least squares algorithm, a rigorous least squares algorithm, an ordinary least squares algorithm, an orthogonal regression algorithm, a logistic regression algorithm, a stepwise regression algorithm, a local regression algorithm, a Multivariate adaptive regression splines (MARS) algorithm, a Locally Estimated Scatterplot Smoothing (LOESS) algorithm, a Locally Weighted Scatterplot Smoothing (LOWESS) algorithm, a Gauss-Newton algorithm, a Levenberg-Marquardt algorithm, a quasi-Newton algorithm, a Davidon-Fletcher-Powell (DFP) algorithm, a Broyden-Fletcher-Goldfarb-Shanno (BFGS) algorithm, a curve-fitting algorithm, an interpolating spline algorithm, or another type of algorithm that may be used to calculate a trendline from multiple data points. The trendline algorithm may also remove statistical outliers and/or include noise filtering calculations.

FIG. 2B is an exemplary chart 240 illustrating an exemplary full battery charge level 241, an exemplary current battery charge level 242, and a distance between these representing electrical charge required 220 from an energy harvester circuit for an exemplary battery at the illustrated current battery charge level to reach the full battery charge level.

The chart 240 indicates that the current battery charge level is lower than the full battery charge level, indicating that the wearable device associated with FIG. 2B has been used for some time after its last battery charge. In particular, the chart indicates that the energy harvesting circuit(s) of the wearable device would need to generate 3198 milliampere-hours (mAh) of electric charge in order to charge the battery of the wearable device back to the full battery charge level. This amount should be understood to be illustrative rather than limiting.

FIG. 2C is an exemplary chart 250 illustrating an exemplary number of burned calories that is extrapolated from the trendline 210 of FIG. 2A to produce the electrical charge required from an energy harvester circuit of FIG. 2B for the battery to reach the full battery level.

In particular, the chart uses the energy value from FIG. 2B (3198 mAh) as a reference point to determine a corresponding calorie amount along the trendline calculated in FIG. 2A from the wearable device's historical data. In this way, the wearable device can tell the user that, based on his/her historical energy generation, the user needs to expend 1274 calories in order to generate 3198 mAh of energy and recharge the device to a full battery charge.

The wearable device can also set less lofty goals, such as simply maintaining the wearable device's current battery charge level, or increasing the wearable device's current battery charge level to a predetermined battery charge level somewhere between the current battery charge level and the full battery charge level. For example, if the current battery charge level is 40%, the wearable device may calculate calories required to increase the wearable device's battery charge level to 75%, which may be more manageable for the user to achieve.

The activities performed by the user associated with these calorie amounts and energy production amounts may be, for example, fitness activities that allow the energy harvesting circuit to produce motion-based energy such as with a piezoelectric energy harvesting circuit or alternatively be heat-based energy. Such fitness activities may include, for example, walking, running, lifting weights, walking with weights, running with weights, jumping, hopping, jumping rope, squatting, swimming, climbing, skiing, snowboarding, skateboarding, bicycling, stretching, doing gymnastics, doing yoga, or playing a sport. Different calorie calculations or algorithms may be associated with different activities in some embodiments.

In various embodiments, the wearable device may have optional wearing positions or locations. Certain wearing positions may increase the energy production of the harvesting circuits on the device thereby providing an advantage in recharge time necessary to recharge the power storage unit. For example, a wearable device position which experiences higher motion swings or a more forceful movement such as on a wrist or ankle, may generate energy quicker as opposed to, for example, a position or location at the waist. Such high movement positions may be more optimal for energy harvesting and the system described herein may recommend one or more wearable device positions leading to the quickest recharge time. Thus, in some aspects, the software of the system for the wearable device may determine a most appropriate location for the highest return on energy harvesting and provide to the user one or more wearable device locations.

In other aspects, the wearable device may be operable to take into account the activity the user would typically do or types of activities recommended and that fit a user's lifestyle or historical activity profile. In such implementation, the wearable device may be operable to advise the wearer a type of activity and/or a location to wear the wearable device. For example, the wearable device may be operable to recommend that the user go running and wear the device around the wrist. Such recommendations could be based upon multiple factors including optimal wearing position for recharging the wearable device, type of activity and expected duration or a wearer/user preference of activities that are available. The device may be operable to not only provide information on the amount of time and calories needed for recharging the power storage unit, but also provide a list of possible activities the wearer could undertake and the time period until full recharge for each activity. Thus, the wearer of the device could make an informed decision as to time necessary until full recharge, type of activity recommended and time to recharge trade-off if the user were to select a less rigorous activity or wearing position.

In other aspects, the wearable device may not have an alternative wearable position but may be configured to determine recharge time required from the at least one energy harvesting circuit if positioned at different places on the user. For example, for recharging purposes the wearable device may be optionally configured to recommend placement of the device in an alternative location, such as placing a wearable device bracelet in a sock, even though placement of the device at such location would result in inaccurate activity measurements but alternatively may result in a quicker recharge time. The system, software and the wearable device may further be configured to allow a user to indicate alternative placement of the wearable device such that during the activity time period, a corrected reading of the device based on similar previous readings for that user and that activity may supplement or replace the readings of the device being placed in an alternative position.

FIG. 3 is a flow diagram illustrating an exemplary operation of a wearable software 300 as executed by an exemplary wearable device.

The exemplary operation may begin with the wearable device polling the clock and triggering running of “routine operations” periodically (e.g., every 60 seconds) at step 301. Next, the wearable device may run its “routine operations”—which may include, for example, obtaining sensor measurements from the health, body, fitness, and environment sensors of the wearable device and storing these sensor measurements in the memory of the wearable device (e.g., at the health sensor database) at step 302. The wearable device may then calculate and store calories used as well as electrical charge generated by the energy harvesting circuit in the memory of the wearable device (e.g., at the historical charging database) at step 303.

Once the historical charging database has enough data points to produce a trendline (also known as the “best fit line”), the wearable device calculates at step 304 the trendline (see e.g., FIG. 2A), which it may then store in the memory of the wearable device (e.g., at the historical charging database). Some embodiments of the wearable device may require two or more sensor-based data points to produce a trendline. Other embodiments may generate a trendline with a single sensor-based data point such as if the wearable device presumes a zero-calories-burned to zero-energy-produced data point. Some alternative implementations may download external data points or external trendlines from external sources when the wearable device is first used, to be gradually supplemented or replaced by the data points and/or trendline based on the user's own historical data.

Once the trendline is calculated, the wearable device determines the currently battery charge level of the battery of the wearable device, and determines an amount of energy needed to charge the battery of the wearable device to a full battery charge level or to a predetermined battery charge level inclusively between the current battery charge level and the full battery charge level (see e.g., FIG. 2B) at step 305. The wearable device then uses this determined amount of energy needed as a reference point to determine, using the trendline, how many calories the user would need to burn to generate that amount of energy (see e.g., FIG. 2C) at step 306.

Once the amount of calories required is calculated at step 307, the wearable device checks the input of the energy harvester GUI (see e.g., FIG. 4) at step 308, extracts frequency settings and battery level settings, and checks the clock and current battery level, respectively, to determine if the requirements of these settings are met. If neither is met, then the operations may return to the beginning of the flow diagram, where the clock is polled to trigger routine operations. If the requirements of the frequency settings or battery level settings from the energy harvester GUI are met, these are used to populate the wearable output GUI (see e.g., FIG. 5) at step 309, and can further be used to execute the wearable output GUI such as by notifying the user that they have reached a calorie/energy/time milestone. If the wearable output GUI settings indicate that a component should be disabled such as to conserve battery power at step 310, then the wearable device may calculate at step 311 energy savings based on the component power database stored in the memory of the wearable device and then calculate, using the trendline, a calorie amount to subtract from the previously calculated calories amount in order to for update the required calorie amount. The wearable device may then update the wearable output GUI at step 312 and the operations may return to the beginning of the flow diagram, where the clock is polled to trigger routine operations.

FIG. 4 illustrates an exemplary energy harvesting graphical user interface (GUI) 180 as executed by an exemplary wearable device. This is a graphical interface that may be displayed at the display of the wearable device and provides an general representation of the various information and related interface of the device.

The first element of the GUI shown is an area for the user to input an energy harvester update frequency 181. The user may select an update frequency using a user interface element (e.g., such as the shown drop-down menu) to select one of the multitude of options. In the exemplary energy harvesting GUI of FIG. 4, the exemplary user selected “hourly,” indicating that the user will receive hourly updates regarding the energy harvesting circuits' progress in recharging the battery. Next, the GUI includes an area for the user to select the battery level 182 which user should be notified. In the exemplary energy harvesting GUI of FIG. 4, the exemplary user selected 18 percent, indicating that once the wearable device's battery level reaches 18%, the user should be notified. The exemplary energy harvesting GUI of FIG. 4 also includes two buttons (e.g., touchscreen buttons or mechanical buttons) which may be pressed by the user to check the calories required to reach a predetermined battery charge level 183, or to cancel out of the energy harvester GUI at 184.

FIG. 5 illustrates an exemplary wearable output graphical user interface (GUI) 190 as executed by an exemplary wearable device. This is a graphical interface that may be displayed at the display of the wearable device.

The first element shown on the exemplary wearable output GUI is an area 191 for the wearable device to display the current charge. The exemplary wearable output GUI of FIG. 5 indicates that the current charge is 18 percent. Next is an area 192 for the wearable device to display the number of calories required to achieve a full battery charge level, or to achieve a predetermined battery charge level. The exemplary wearable output GUI of FIG. 5 indicates that 1,274 calories has been calculated (based on the trendline) as the number of calories that the user should burn in order to recharge the wearable device's batter to the predetermined battery charge level. The exemplary wearable output GUI may also include, for example, a motivational message for the user. The exemplary wearable output GUI of FIG. 5, for example, tells the user “you can do it!” to provide motivation.

Below these areas of the exemplary wearable output GUI is an area where the user can select one or more components of the wearable device to disable in order to conserve power. This may be done, for example, through a drop-down menu 193, radio button list, checkbox list, grid, or similar interface in which a user may select one or more components of the wearable device to disable. For example, the exemplary wearable output GUI of FIG. 5 allows the user to disable the radio (e.g. from the communications module) of the wearable device, a pulse oximeter sensor (e.g., of the sensors 1-N) of the wearable device, the display of the wearable device, or a thermometer sensor (e.g., of the sensors 1-N) of the wearable device. The exemplary wearable output GUI of FIG. 5 indicates that the exemplary user has selected to disable the pulse oximeter sensor at 194.

In some embodiments the list of sensors set forth in the menu may also include a user priority setting list based upon what information may be derived from the sensors. Such priority list and recommendation provided by the system may allow a more intelligent mechanism of determining which sensors should remain on during the activity and which can be off. Thus, for some health goals, a combination of sensors may be required during the user activity at differing points in the activity. For example, an accelerometer may be necessary to measure exercise but may be turned off immediately after the exercise has ceased. However, immediately after the activity has ceased, proper activity analysis and recordation of health goals may require automatically turning on the heart rate and blood pressure sensors to measure recovery information. Further, during the activity at various points the user may want to turn on the temperature sensor and respiratory rate to detect over exertion or adjust exercise coaching advice on the go. Thus, a priority list of sensors may be provided based upon user or activity preference as well as health goals thereby requiring activation of sensors at various times. The list of sensors provided may incorporate such priority list so that the user may operatively select, in various embodiments, activation and disabling of sensors at various times or based upon necessary information obtain by the listed sensor.

A test screen button 195 is provided for the user to update the number of calories to achieve a full charge because of the disabled component or because of the user's fitness activity and/or calories burned since the last update. Finally, there is a button for the user to return to the energy harvester GUI (see e.g., FIG. 4).

An exemplary use case of the exemplary wearable device pictured in FIG. 4 and FIG. 5 is provided. Using the exemplary wearable device of FIG. 2, a user of the wearable might set up power level for checking the battery and an update frequency. In this example, the user has selected 18% and hourly, respectively.

Previously, the device has recorded charge information and calorie information as the device has been used in various fitness or other activities and stored such in formation in the historical charging database. The system and method may determine, based on that data, a trendline or best fit line in order to predict the number of calories required to harvest a certain number of mAh of charge. The trendline may be modeled as an equation or as a set of equations if the trendline is modeled by local segments. In this exemplary use case, an exemplary trendline equation may be:

Calories Required=(0.35)*(energy in mAh)+155 calories.

When the user requests a calorie check using the button in the exemplary wearable output GUI of FIG. 5, the device determines its current charge level of 702 mAh and calculates the difference between that and the maximum charge. Assuming a 3900 mAh battery wherein a full battery charge level is 3900 mAh, the difference would be 3198 mAh. The device then calculates the number of calories the user will need to burn to generate sufficient charge so that the device is fully charged. In this example, the user would need to burn 1274 calories to be fully recharged.

After checking to make sure the frequency and battery settings from the energy harvester GUI are met, the device prompts the user to select any components he wishes to disable to conserve power. A user might select, for example, to disable the pulse oximeter sensor to reduce power (see e.g., FIG. 5). The device then calculates energy savings from component power database using the trendline (“line of best fit”)—here 58 mAh and then calculates using best fit line for component calories, 175 calories, and subtracts that value from final calories for updated final calories.

In some embodiments, the wearable device may return charge and calorie values that maximize the life of the battery.

In some embodiments, the wearable device interacts with calorie or context data, and takes into account projected calorie burn/energy harvesting to forecast future charge levels. For instance, the wearable device can take into account historical use, and project/forecast to the user when the wearable device will run out of power/reach certain battery charge level minimums predetermined by the user, the wearable device, or a third party.

In some embodiments, the wearable device can suggest a type of exercise such as walking, running, lifting weights, walking with weights, running with weights, jumping, hopping, jumping rope, squatting, swimming, climbing, skiing, snowboarding, skateboarding, bicycling, stretching, doing gymnastics, doing yoga, or playing a sport and a timed duration of the suggested exercise to burn the required number of calories to charge the battery using the energy harvesting circuits.

In some embodiments, the trendline and/or historical trendlines, since the trendline may change as more historical data is accrued can be sent to network as data that can be used to help new users see community activities, that is, those that have never used the battery-charging-via-calorie-burn technology, to help guide estimates for those users. This data can also be shared between users so as to create a community competition.

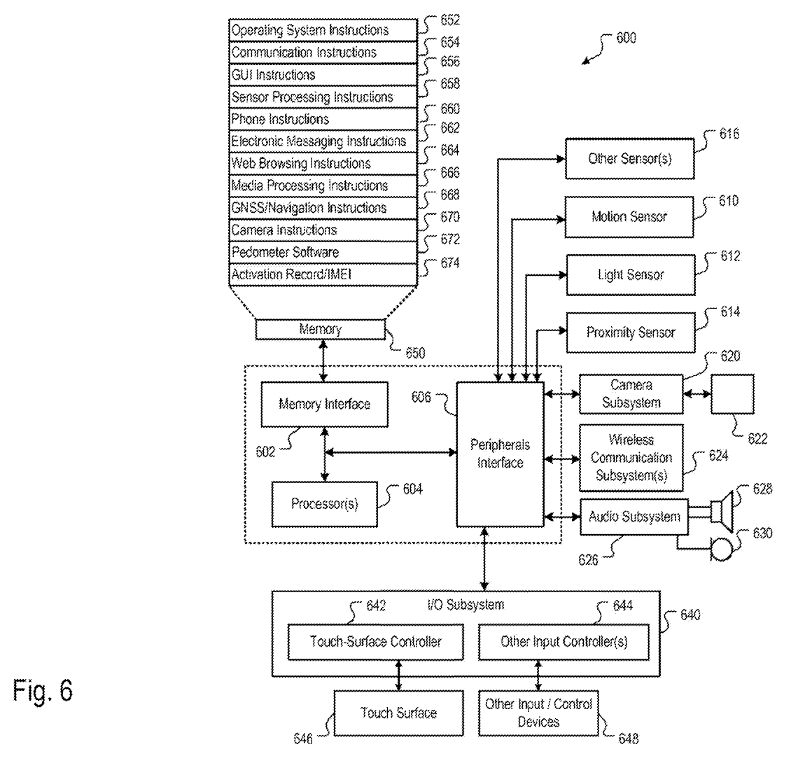

FIG. 6 illustrates an exemplary computing device architecture that may be utilized to implement the various features and processes described herein. For example, the computing device architecture 600 could be implemented in the wearable device. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 is meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, and communications capability.

FIG. 7 illustrates an exemplary historical charging database 160 that may be stored in the memory of an exemplary wearable device.

As shown in the implementation of the FIG. 7, the historical charging database may incorporate date and time of the reading. The third element may indicate the energy developed by the energy harvesting circuit(s) at/by that date and time. The fourth element may indicate a calculation for calories burned by the user based on sensor readings of the wearable device, such as accelerometer or pulse sensor readings while generating the energy via the energy harvesting circuit(s).

The database may also store the trendline, for example, as an equation. The exemplary historical charging database includes an exemplary trendline equation in which is calorie used is equal to 0.35 milliamp hours times energy plus 155 calories.

FIG. 8 illustrates an exemplary component power database 150 that may be stored in the memory of an exemplary wearable device. This database shows power usage of various components of the wearable device. These numbers may come preinstalled with the wearable device (i.e., input by the manufacturer or vendor prior to sale), they may be downloaded/updated from the internet (i.e., from a manufacturer/vendor web portal, or from another user, or based on average power usage of such components for a set of multiple users) or they may be determined by the wearable device (e.g., using a built-in multimeter component) (e.g., determined once, periodically updated, or averaged through periodic checks over time).

The exemplary component power database of FIG. 8 lists power consumption of an exemplary accelerometer (3.8 mAh), an exemplary thermometer (11 mAh), an exemplary display (30 mAh), an exemplary radio (e.g., from the communications module) (160 mAh), and an exemplary pulse oximeter (58 mAh).

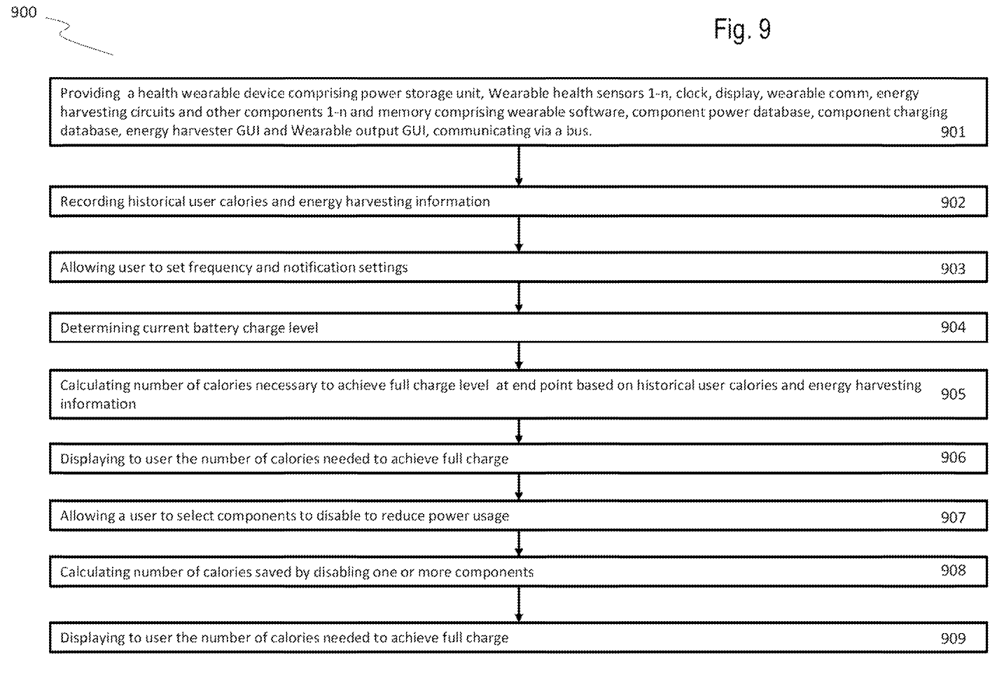

FIG. 9 illustrates an exemplary overall method of the present disclosure and embodiments as described herein.

The overall method may include at step 901 providing a wearable device as described herein, including one or more wearable device health, body, fitness environment sensors 1-n, a clock, a power storage unit, one or more energy harvesting circuits, a display, a memory, a communication module (“wearable comm”), and a variety of other components 1-n. These components may be communicatively coupled at a single bus, or may alternatively be connected in a more disjointed manner. The memory of the wearable device may include a wearable software (see e.g., FIG. 3), a component power database (see e.g., FIG. 8), a historical charging database (see e.g., FIG. 7), a health sensor database, an energy harvester graphical user interface (“GUI”) (see e.g., FIG. 4), a wearable output GUI (see e.g., FIG. 5), and various other software elements.

The overall method may include at step 902 recording historical user calorie burn data and historical energy harvesting data. The overall method may include at step 903 allowing the user to set frequency and notification settings. The overall method may include at step 904 determining a current battery charge level.

The overall method may include at step 905 calculating a number of calories necessary to achieve a full battery charge level or a predetermined battery charge level at end point based on a trendline extrapolated from the historical user calories and energy harvesting information.

The overall method may include at step 906 displaying to user the number of calories needed to achieve full charge. The overall method may include at step 907 allowing a user to select components to disable to reduce power usage. The overall method may include at step 908 calculating number of calories saved by disabling one or more components. The overall method may include at step 909 displaying to user the number of calories needed to achieve the full battery charge level or the predetermined battery charge level.

While the flow diagram in FIG. 9 shows a particular order of operations performed by certain embodiments of the disclosure, it should be understood that such order is exemplary as alternative embodiments can perform the operations in a different order, combine certain operations, overlap certain operations, etc.

Embodiments of the present disclosure also relate to an apparatus for performing the operations herein. Such a computer program is stored in a non-transitory computer readable medium. A machine-readable medium includes any mechanism for storing information in a form readable by a machine such as a computer. For example, a machine-readable and computer-readable medium includes a machine such as computer readable storage medium (e.g., read only memory (“ROM”), random access memory (“RAM”), magnetic disk storage media, optical storage media, flash memory devices).

The processes or methods depicted in the preceding figures can be performed by processing logic that comprises hardware (e.g. circuitry, dedicated logic, etc.), software (e.g., embodied on a non-transitory computer readable medium), or a combination of both. Although the processes or methods are described above in terms of some sequential operations, it should be appreciated that some of the operations described can be performed in a different order. Moreover, some operations can be performed in parallel rather than sequentially.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A method, comprising:
calculating by one or more processors a trendline based on historical data stored in a memory of a wearable device, the historical data including a plurality of calorie amounts, where each of the calorie amount of the plurality of calorie amounts is calculated based on one or more sensor measurements generated by one or more sensors of the wearable device during a sensed time period, and each calorie amount of the plurality of calorie amounts is also associated with an energy amount, the energy amount indicating an amount of energy generated by an energy harvesting circuit during the calorie amount's sensed time period;
determining, by one or more of the processors, a current battery charge level of a battery of the wearable device;
determining, by one or more of the processors, a charge difference indicating a required amount of electric charge from the energy harvesting circuit to increase the current battery charge level to a predetermined battery charge level of the battery of the wearable device, wherein the predetermined battery charge level is inclusively between the current battery charge level and a full battery charge level of the battery of the wearable device;
calculating, by one or more of the processors, a calorie requirement indicating a number of calories that, according to the trendline, should generate the charge difference through the energy harvesting circuit; and
generating an alert based on the calorie requirement.

2. The method of claim 1 further including
presenting a list of selectable sensors to a user on a display;
determining a selection by the user of at least one of the selectable sensors to disable;
reducing the calculated calorie requirement by a value related to the disabled selected sensor and providing the reduced calculated calorie requirement on the display.

3. A system, comprising:
a wearable device having at least one sensor;
the wearable device further having at least one energy harvesting circuit electrically connected to a power storage unit; and
at least one processor connected to a memory and having instructions configured to:
determine calories used and energy developed during a determined time period based upon information received from the at least sensor and the at least one energy harvesting circuit;
record the determined calories used and energy developed in a historical charging database;
generate a best fit line from the historical charging database;
determine a power charge level for the power storage unit of the wearable device;
determine an amount of energy needed to charge the power storage unit to a predetermined level based on the best fit line, power charge level; and
provide the determined amount of energy needed to a display.

4. The system of claim 3 wherein the processor is further configured to:
present to the display a selectable list of the at least one sensor;
receive instructions to disable at least one of the sensors; and
determine energy savings from the at least one disabled sensor.

5. The system of claim 4 wherein the processor is further configured to present the determined energy savings to the display.

6. The system of claim 3 wherein the at least one sensor is a plurality of sensors configured to monitor health parameters of a wearer of the wearable device.

7. The system of claim 3 wherein the at least one energy harvesting circuit is configured to generate energy during physical activity of a wearer of the wearable device.

8. The system of claim 3 wherein the at least one energy harvesting circuit is configured to generate energy from motion the wearable device.

9. A method or providing energy harvesting information for a wearable device, comprising:
determining calories burned by a wearer of the wearable device and energy developed during a determined time period based upon information received from at least one sensor on the wearable device and at least one energy harvesting circuit on the wearable device;
determining a power charge level for a power storage unit of the wearable device;
calculating, based on the calories used and energy developed during the determining time period, an amount of calories the wearer needs to burn in order to charge the power storage unit from the determined power charge level to a predetermined level; and
displaying the determined amount of calories needed.

10. The method of claim 9 further comprising:
saving the calories used and the energy developed during the predetermined time period.

11. The method of claim 10 further including calculating the amount of calories the wearer needs to burn to charge the power storage unit based upon the saved calories used and energy developed.

12. The method of claim 9 wherein the calculating the amount of calories the wearer needs to burn is based upon past activity of the wearer of the wearable device.

13. The method of claim 11 wherein the calculating the amount of calories the wearer needs to burn is based upon a trendline created from the saved calories used and energy developed.

14. The method of claim 9 wherein the calculating the amount of calories the wearer needs to burn is based upon the wearer's current activity level.

15. The method of claim 9 wherein the calculating the amount of calories the wearer needs to burn is based upon the wearer's historical activity level.

16. The method of claim 9 further including:
presenting to a display of the wearable device a selectable list of the at least one sensor;
receiving instructions to disable at least one of the sensors;
determining energy savings from the at least one disabled sensor; and
calculating a second amount of calories the wearer needs to burn needed to charge the power storage unit to the predetermined level.

17. The method of claim 16 further including presenting to the display the second amount of calories the wearer needs to burn.

18. The method of claim 16 further including presenting to the display a time period related to generating the second amount of calories the wearer needs to burn.

19. A wearable device, comprising:
at least one sensor;
at least one energy harvesting circuit electrically connected to a power storage unit; and
at least one hardware processor connected to a memory and having instructions configured to:
determine calories used and energy developed during a determined time period based upon information received from the at least sensor and the at least one energy harvesting circuit;
record the determined calories used and energy developed in a historical charging database;
generate a best fit line from the historical charging database;
determine a power charge level for the power storage unit of the wearable device;
determine an amount of energy needed to charge the power storage unit to a predetermined level based on the best fit line, power charge level; and
provide the determined amount of energy needed to a display.

20. The wearable device of claim 19 wherein the processor is further configured to:
present to the display a selectable list of the at least one sensor;
receive instructions to disable at least one of the sensors; and
determine energy savings from the at least one disabled sensor.

21. The wearable device of claim 20 wherein the processor is further configured to present the determined energy savings to the display.

22. The wearable device of claim 19 wherein the at least one sensor is a plurality of sensors configured to monitor health parameters of a wearer of the wearable device.

23. The wearable device of claim 19 wherein the at least one energy harvesting circuit is configured to generate energy from motion of the wearable device.

24. The wearable device of claim 23 wherein the at least one energy harvesting circuit is configured to generate energy during physical activity of a wearer of the wearable device.
